(19) United States
(12) Patent Application Publication
(10) Pub. No.: US 2024/0013860 A1
(43) Pub. Date: Jan. 11, 2024

(54) METHODS AND SYSTEMS FOR PERSONALIZED NEOANTIGEN PREDICTION

(71) Applicant: BIOINFORMATICS SOLUTIONS INC., Waterloo (CA)

(72) Inventors: Hieu TRAN, Waterloo (CA); Ming LI, Waterloo (CA)

(21) Appl. No.: 18/347,105

(22) Filed: Jul. 5, 2023

Related U.S. Application Data
(60) Provisional application No. 63/358,573, filed on Jul. 6, 2022.

(51) Int. Cl. G16B 20/30; G16B 20/20; G01N 33/68

(52) U.S. Cl. CPC G16B 20/30; G16B 20/20; G01N 33/6878

(57) ABSTRACT

Personalized machine learning systems and methods are provided to predict the collective response of a patient's CD8+ T cells by modeling positive and negative selection processes. For each individual patient, HLA-I self peptides were used as negative selection, and allele-matched immunogenic T cell epitopes as positive selection. The negative and positive peptides were used to train a binary classification model, which was then applied to predict the immunogenicity of candidate neoantigens of that patient.

### CROSS-REFERENCE TO RELATED APPLICATION

The present application claims the benefit of U.S. Provisional Application No. 63/358,573 filed on Jul. 6, 2022, titled “Methods and Systems for Personalized Neoantigen Prediction”.

### FIELD

The present disclosure generally relates to personalized identification of neoantigens and in particular computer-implemented methods and systems based on positive and negative selections.

### BACKGROUND

Tumor-specific human leukocyte antigen (HLA) peptides are only expressed on the surface of cancer cells and hence represent ideal targets for the immune system to distinguish cancer cells from normal cells. However, due to the high specificity of T cells, only a small fraction of those peptides can be recognized by T cells to trigger immune responses. They are referred to as neoantigens. Despite their promising role in cancer immunotherapy, the chance of finding neoantigens is remarkably low, usually less than half a dozen out of thousands of somatic mutations detected per patient. Thus, in silico methods are essential to accurately predict and prioritize candidate neoantigens before in vitro validations, which often involve time-consuming and costly experiments. A typical approach in existing studies is to predict a limited number of candidate neoantigens, e.g. ˜20, and then synthesize and test them in vitro for immunogenicity, with the hope that 2-3 candidates show positive responses. Hence, improved methods are needed.

### SUMMARY

In one aspect, there is provided a computer-implemented method for personalized identification of neoantigens from sample peptides sequences obtained from a patient, the method comprising: obtaining a first dataset of HLA-1 binding self peptides sequences of the patient; obtaining a second dataset of patient allele-matched T-cell epitope sequences; wherein the first and second datasets are for training an artificial neural network to classify the sample peptide sequences based on T-cell recognition; selecting sample peptide sequences that match with sequences of the second dataset; and excluding sample peptide sequences that match with the first dataset, wherein the remaining selected sample peptide sequences are identified as candidate neoantigens.

In another aspect, there is provided a computer implemented system for personalized identification of neoantigens from sample peptides sequences obtained from a patient using neural networks, the computer implemented system comprising: a processor and at least one memory providing a plurality of layered nodes configured to form an artificial neural network for generating a probability measure for one or more neoantigen candidates, the artificial neural network trained on: a first dataset of HLA-1 binding self peptides of the patient, and a second dataset of patient allele-matched T-cell epitopes, to classify the sample peptide sequences based on T-cell recognition, wherein the plurality of layered nodes receives a peptide sequence as input; the processor configured to: select sample peptide sequences that match with sequences of the second dataset, and exclude sample peptide sequences that match with the first dataset, wherein the remaining selected sample peptide sequences are identified as candidate neoantigens.

In this respect, before explaining at least one embodiment in detail, it is to be understood that the embodiments are not limited in application to the details of construction and to the arrangements of the components set forth in the following description or illustrated in the drawings. Also, it is to be understood that the phraseology and terminology employed herein are for the purpose of description and should not be regarded as limiting.

Many further features and combinations thereof concerning embodiments described herein will appear to those skilled in the art following a reading of the instant disclosure.

### DESCRIPTION OF THE FIGURES

Embodiments of devices, apparatus, methods, and kits are described throughout reference to the drawings.

FIG. 1 shows a Predicting the immunogenicity of candidate neoantigens by modeling the central tolerance of CD8+ T cells in each individual patient. (A) The positive and negative selection processes, i.e. the central tolerance of CD8+ T cells. (B) Personalized model training to resemble the central tolerance of CD8+ T cells in an individual patient. (APC: Antigen-Presenting Cell; HLA: Human Leukocyte Antigen; MS: Mass Spectrometry; IEDB: Immune Epitope Database).

FIG. 2 shows a bar graph of performance evaluation of four immunogenicity prediction tools on three individual patients and one mouse cancer cell line. Y-axis indicate Area Under the ROC Curve (AUC) of the prediction tools on experiment-validated neoantigens. X-axis indicate the cell lines used, with each set of four bars representing the AUC of the prediction tools DeepImmun, PRIME, NETMHCpan, and IEDB presented in said order. (IEDB refers to the immunogenicity prediction tool on IEDB website developed by Calis et al., 2013).

FIG. 3 shows a block diagram of an example computing device.

### DETAILED DESCRIPTION

Majority of current in silico methods for predicting candidate neoantigens focus on predicting the binding affinity of class 1 and class 2 peptide-HLA (pHLA) complexes or, given the rise of recent mass spectrometry (MS) based immunopeptidomics, the entire pHLA presentation pathways. The peptides are then ranked by their HLA binding/presentation scores, their expression levels, and other criteria, based on the assumption that if a mutated peptide is presented in large amounts on the cancer cell surface, it has a better chance to trigger T cell responses. However, such approaches only consider half of the equation and do not take into account how a pHLA complex can be recognized by T cell receptors (TCRs). Accordingly, systems and methods that focus on or are trained to predict the binding affinity of a peptide (such peptide-MHC interaction) have limited applicability. Early studies on HLA class 1 (HLA-I) T cell epitopes have suggested that some amino acid positions (e.g. 4-6) of the epitopes were in close contact with the TCRs, while the anchor positions (e.g. 1, 2, 9) were responsible for HLA-I binding. Recently Schmidt et al. proposed PRIME, a model that simultaneously predicts the HLA-I binding and the TCR recognition of a peptide by specifically assigning which of its amino acid positions are responsible for each task. In addition, certain properties of amino acid residues, such as hydrophobicity, polarity, or large and aromatic side chains, were found to have statistically significant correlation with the epitope immunogenicity. Sequence similarity to known pathogen epitopes and dissimilarity to the human proteome have also been proposed for immunogenicity prediction.

However, the above methods fall short of accounting for the high specificity of each individual patient's T cells. Of note is that both cancer and immunotherapy are associated with a great amount of personal genetic variations of each individual patient. The HLA complex, the T cell population, and the tumor mutations themselves are significantly different from one patient to another. Thus, cancer immunotherapy should strongly benefit from personalized approaches. A personalized model for T cell immunogenicity prediction is needed. One approach is to sequence the TCRs of a patient and predict the TCR-pH LA recognition from their sequences or structures. However, despite on-going intensive efforts of TCR sequencing, it is difficult to sequence the complete TCR repertoire for every patient. Thus a personalized model for predicting TCR-pHLA recognition with minimal sequencing the TCRs is needed.

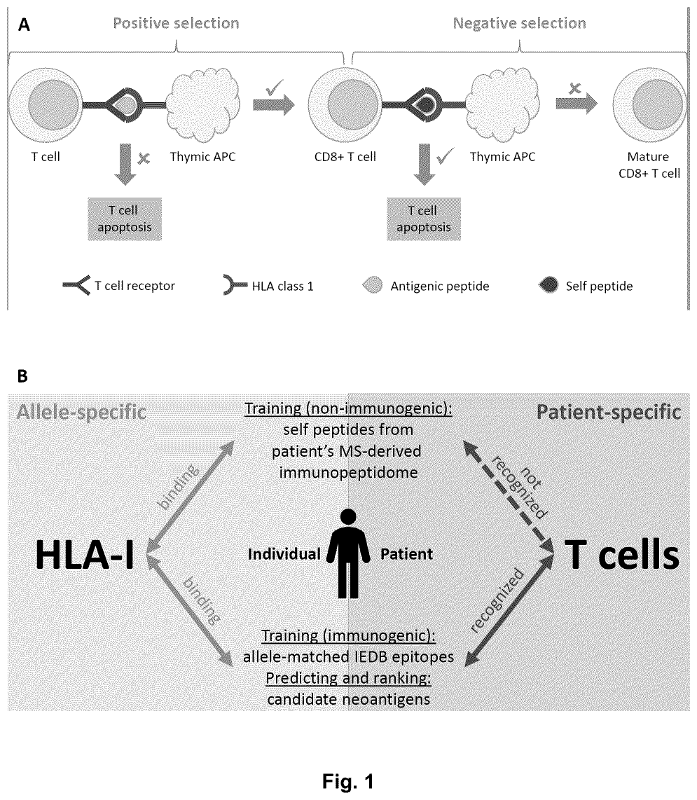

In accordance with the present disclosure, a personalized machine learning approach is provided that predicts the collective response of a patient's CD8+ T cells by modeling the positive and negative selection processes. The specificity of T cells are mainly shaped by the positive and negative selection processes, i.e. the central tolerance that happens inside the thymus of each individual patient (see FIG. 1, panel A). During the positive selection, T cells are selected by their ability to bind to pHLA complexes: they will become CD8+ or CD4+ T cells if they bind to HLA-I or HLA-II, respectively; otherwise they will die by neglect. During the negative selection, T cells are selected against their ability to bind to self peptides: those that have high affinity for self peptides will die by clonal deletion, preventing the risk of autoimmunity; the remaining will mature and participate in immune responses against foreign antigens. Extensive efforts, both experimental and computational, have been put into modeling complicated molecular mechanisms behind those processes.

The methods and systems provided herein utilizes the positive and negative selections encapsulated by the complete space of positive and negative peptides resulting from those selections. In some embodiments, MS-based immunopeptidomics is used to obtain HLA-I self-peptides to resemble the negative selection of CD8+ T cells in an individual patient. It was discovered that focusing on HLA-I and CD8+ and not HLA-II and CD4+ is advantageous because of data availability and the corresponding complexity of immunopeptidomics assays. Since positive selection is not entirely personalized but rather HLA-dependent, all epitopes reported in positive T cell assays can be collected from databases such as the Immune Epitope Database (IEDB) that match the patient's HLA-I alleles. Hence in one particular example, for each individual patient, his/her HLA-I self peptides derived from mass spectrometry-based immunopeptidomics was collected as negative selection, and allele-matched immunogenic T cell epitopes were collected from the Immune Epitope Database as positive selection.

Using this personal dataset of positive and negative peptides, binary classification model was trained specifically for that patient to predict his/her T cell response to any given peptide. The model allows for prediction of the immunogenicity of candidate neoantigens for that patient. It's worth emphasizing that, while previous studies have used immunogenic and non-immunogenic peptides to train prediction models, the peptides were collected from existing literatures or databases in an HLA-allele-specific manner. It was discovered that using self peptides, preferably derived from MS-based immunopeptidomics, of an individual patient as non-immunogenic for that particular patient allows for improved and personalized prediction and identification of neoantigens. In particular, by using de novo peptides sequencing from mass spectrometry data, this removes the reliance or at least removes some of the reliance on existing literatures or databases.

In the description that follows, a number of terms conventionally used are utilized. In order to provide a clear and consistent understanding of the specification and claims, and the scope to be given to such terms, the following definitions are provided.

The terms “subject,” “individual,” and “patient” are used interchangeably herein to refer to a mammal being diagnosed or assessed for treatment and/or being treated. The terms “subject,” “individual,” and “patient” encompass, without limitation, individuals having or at risk of having cancer or tumor growth. Subjects may be human, but also include other mammals, particularly those mammals useful as laboratory models for human disease, e.g. mouse, rat, etc.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

As used herein, the term “match with” or “similar to” and like terms when comparing protein sequences, refers to having in part or whole common amino acid sequences or having in part or whole corresponding sequences that result in protein structure having equivalent or partial biological activity or function, or protein binding.

In accordance with the present disclosure, computer-implemented systems and methods are provided for personalized identification of neoantigens from sample peptides sequences obtained from a patient. In one embodiment, candidate neoantigens are tumour antigens. The computer-implemented systems and methods involves training an artificial neural network trained on at least two datasets, each containing peptide sequences. For example, a dataset may comprise a table, a matrix, a vector, a string, or a list of peptide sequences (i.e. in the form of amino acid name, single letter code, multiple letter code, or integer indices).

The first dataset comprises amino acid sequences of HLA-1 binding self peptides of the patient for whom neoantigen identification and/or diagnosis is provided. As used herein “HLA-1 binding self peptides” refers to peptides obtained from said patient that bind to HLA-1 molecules of said patient. HLA-1 molecules are encoded by the human leukocyte antigen class 1 (HLA-1) genes (i.e. HLA-A, HLA-B, and HLA-C genes), or fragments thereof. Since the computer-implemented systems and methods described herein are trained using these HLA-1 binding self peptides from each individual patient, the systems and methods allow for personalized identification of neoantigens.

In some embodiments, peptides obtained from the patient were identified as HLA-1 binding self peptides of a patient by conducting HLA-I immunoprecipitation assays on a sample (such as cell sample) from said patient. In one embodiment, the patient cell sample is a normal cell sample. In other embodiments, the patient cell sample is a combination of normal and tumor cells sample. Exemplary immunoprecipitation assays are within the purview and readily appreciated by a skilled person in the art. The identified HLA-1 binding self peptides are then sequenced. In one embodiment, the HLA-1 binding self peptides are then sequenced using protein mass spectrometry. Exemplary systems and methods for protein sequencing using mass spectrometry are disclosed in US Publication Nos. US20190018019, US20190147983, US20200243164, or US20200326348, the entire content of which is incorporated herein by reference.

The second dataset comprises amino acid sequences of patient allele-matched T-cell epitope sequences. A T-cell epitope is a part of an antigen that is recognized by T cells. As used herein, a “patient allele-matched T-cell epitope” refers to an epitope that is recognized by a T cell and that match the patient's HLA-I alleles. In some embodiments, T-cell epitope sequences were obtained by conducting T cell assays. In some embodiments, T-cell epitope sequences were obtained from a database. T-cell epitope sequences were then selected for the second dataset by matching against the patient's HLA-1 alleles.

In some embodiments, the first and/or second dataset comprises amino acid sequences that are between 5 and 20 amino acids in length, preferably between and including 8 and 14 amino acids in length.

The first and second datasets are used to train a classification model, preferably a binary classification model, specifically for said patient to predict his/her T cell response to a given peptide. The computer-implemented systems and methods described herein receive sample peptide sequences from the patient as input and are configured to select sample peptide sequences that match with sequences of the second dataset. Sample peptide sequences that match with the first dataset are excluded. The remaining selected sample peptide sequences, that were not excluded, are identified as candidate neoantigens. In some embodiments, the systems and methods described herein output peptide sequences that have been identified as candidate neoantigens. In some embodiments, the systems and methods described herein output a probability score representing the likelihood that a candidate neoantigen will be recognized by CD8+ T cells of the patient.

In some embodiments, the system described herein comprise a processor and at least one memory providing a plurality of layered nodes configured to form an artificial neural network. In some embodiments, the plurality of layered nodes comprise an embedding layer. In one embodiment, the plurality of layered nodes comprise an embedding layer of 8 neural units. In some embodiments, the plurality of layered nodes comprise a bi-directional LSTM layer. In one embodiment, the plurality of layered nodes comprise a bi-directional LSTM layer of 8 units. In some embodiments, the plurality of layered nodes comprise a fully-connected layer with L2 regularizer. In some embodiments, the plurality of layered nodes comprise a sigmoid activation layer.

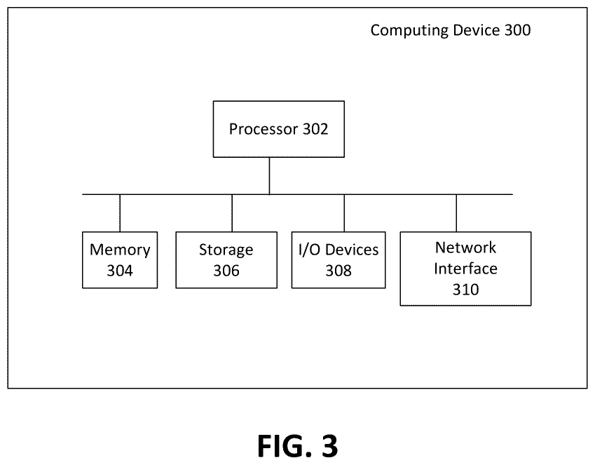

FIG. 3 is a block diagram of an example computing device 300 configured to perform one or more of the aspects described herein. Computing device 300 may include one or more processors 302, memory 304, storage 306, I/O devices 308, and network interface 310, and combinations thereof. Computing device 300 may be a client device, a server, a supercomputer, or the like.

Processor 302 may be any suitable type of processor, such as a processor implementing an ARM or x86 instruction set. In some embodiments, processor 302 is a graphics processing unit (GPU). Memory 304 is any suitable type of random access memory accessible by processor 302. Storage 306 may be, for example, one or more modules of memory, hard drives, or other persistent computer storage devices.

I/O devices 308 include, for example, user interface devices such as a screen including capacity or other touch-sensitive screens capable of displaying rendered images as output and receiving input in the form of touches. In some embodiments, I/O devices 308 additionally or alternatively include one or more of speakers, microphones, sensors such as accelerometers and global positioning system (GPS) receivers, keypads, or the like. In some embodiments, I/O devices 308 include ports for connecting computing device 300 to other computing devices. In an example embodiment, I/O devices 308 include a universal serial bus (USB) controller for connection to peripherals or to host computing devices.

Network interface 310 is capable of connecting computing device 300 to one or more communication networks. In some embodiments, network interface 310 includes one or more or wired interfaces (e.g. wired ethernet) and wireless radios, such as WiFi, Bluetooth, or cellular (e.g. GPRS, GSM, EDGE, CDMA, LTE, or the like). Network interface 310 can also be used to establish virtual network interfaces, such as a Virtual Private Network (VPN).

Computing device 300 operates under control of software programs. Computer-readable instructions are stored in storage 306, and executed by processor 302 in memory 304. Software executing on computing device 300 may include, for example, an operating system.

The systems and methods described herein may be implemented using computing device 300, or a plurality of computing devices 300. Such a plurality may be configured as a network. In some embodiments, processing tasks may be distributed among more than one computing device 300.

Numerous details have been set forth to provide an understanding of the examples described herein. The examples may be practiced without these details. The description is not to be considered as limited to the scope of the examples described herein.

### EXAMPLES

The following examples illustrate certain embodiments addressing specific design requirements and are not intended to limit the embodiments described elsewhere in this disclosure.

### Example 1—Results

FIG. 1 panel B describes the training/evaluation data for each individual patient and trained a personalized model to predict the response of his/her CD8+ T cells.

Negative (non-immunogenic) training data: HLA-I self peptides from MS-based immunopeptidomics. For each individual patient, HLA-I peptides were retrieved from HLA-I immunoprecipitation assays followed by MS experiments on the patient's sample. The MS data was searched against the standard Swiss-Prot human protein database using PEAKS Xpro with no-enzyme-specific cleavage and false discovery rate (FDR) of 1%. The identified peptides were subsequently checked for their HLA-I characteristic length distribution; those with lengths <8 or >14 amino acids were filtered out. The resulting peptides were considered as HLA-I self peptides of the patient and not recognized by the patient's T cells (i.e. non-immunogenic).

Positive (immunogenic) training data: allele-matched IEDB epitopes. The table of T cell assays were downloaded from IEDB with the following filters: linear epitopes, HLA class I, and host as human or mouse. For each individual patient, epitopes were selected that matched his/her HLA-I alleles and had positive Assay Qualitative Measure (including Positive, Positive-High, Positive-Intermedia, Positive-Low). Epitopes with lengths <8 or >14 amino acids were filtered out. The resulting peptides were assumed to be recognized by the patient's T cells (i.e. immunogenic).

Experimentally validated neoantigens for evaluation. To accurately evaluate the personalized model of each individual patient, it is essential to use the neoantigens that had been validated by T cell assays on that same patient. Usually there were a few dozens of candidate neoantigens that were experimentally tested per patient, with less than half a dozen showing positive T cell responses and the rest showing negative. Note that those candidate neoantigens had been carefully excluded from the patient's training data before the training process.

Performance evaluation of immunogenicity prediction tools. As described above, the personalized approach described herein required the personal data of each individual patient for training and evaluation. In particular, HLA-I self peptides from MS-based immunopeptidomics are needed as negative training data and experimentally validated neoantigens are needed for evaluation; both types of data have to come from the same patient. This kind of individual patient's data is limited and four such datasets were found from the literature, including three cancer patients and one mouse cancer cell line: Mel-15, Mel-0D5P, Mel-51, and Mouse-EL4. A summary of those datasets is presented in Table 1. The number of HLA-I self peptides varied from 746-35548 per patient, while the number of immunogenic epitopes varied from 304-2417. The number of candidate neoantigens tested in T cell assays varied from 7-152 and the number of immunogenic neoantigens varied from 2-5 per patient. The raw data of full lists of peptides for training and evaluation not included.

TABLE 1
Performance evaluation of four immunogenicity prediction tools on three
individual patients and one mouse cancer cell line with a summary
of HLA alleles, training and evaluation data of four patients.

| | Training peptides | | Training neoantigens | | |
|---|---|---|---|---|---|
| Patient | Positive | Negative | Positive | Negative | HLA alleles |
| Mel-15 | 304 | 35548 | 5 | 23 | HLA-A03:01; HLA-A68:01; HLA-B27:05 |
| | | | | | HLA-B35:03; HLA-CO2:02; HLA-C04:01 |
| Mel-0D5P | 497 | 10447 | 4 | 148 | HLA-A01:01; HLA-A23:01; HLA-807:02 |
| | | | | | HLA-B15:01; HLA-C12:03:HLA-C14:02 |
| Mel-51 | 2417 | 23691 | 4 | 11 | HLA-A01:01; HLA-A02:01:HLA-814:02 |
| | | | | | HLA-B15:01; HLA-C03:04; HLA-C08.02 |
| Mouse-EL4 | 1907 | 746 | 2 | 5 | H2-Db; H2-Kb |

Mel-15 is a melanoma patient dataset first published by Bassani-Sternberg et al. Bassani-Sternberg et al. and Wilhelm et al. identified 28 mutated HLA-I peptides from the MS and RNA-seq data of native tumor tissues of the patient. The peptides were subsequently tested against the patient's own T cells and five of them showed positive responses (Table 1). Mel-5D0P is a melanoma patient dataset first published by Chong et al. The authors used an MS-based proteogenomic approach to identify non-canonical HLA peptides from tumor samples. They identified 152 HLA-I peptides from long non-coding genes, transposable elements, alternative open reading frames, and tumor-associated antigens; four of them showed positive responses against the patient's own T cells. Mel-51 dataset was obtained from Kalaora et al., who investigated bacteria-derived HLA peptides from melanoma samples. 15 recurrent bacteria-derived HLA-I peptides were identified from patient Mel-51, four of them showed positive T cell responses. The murine cancer cell line Mouse-EL4 dataset was obtained from Laumont et al., who used an MS-based proteogenomic approach to investigate non-coding regions to identify tumor-specific antigens. The authors identified seven tumor-specific peptides, including five aberrantly expressed and two mutated; two of them showed positive T cell responses.

For performance evaluation, the present method was compared to three leading tools, including PRIME, NetMHCpan, and IEDB immunogenicity predictor. NetMHCpan is one of the earliest and most common tools for HLA-I binding prediction. IEDB immunogenicity predictor is one of the earliest HLA-I immunogenicity prediction tools that showed the association between immunogenicity and amino acid positions 4-6, plus other properties of amino acid residues, such as hydrophobicity, polarity, or large and aromatic side chains. PRIME is a recent immunogenicity prediction tool that simultaneously models HLA-I binding and TCR recognition. It should be noted that NetMHCpan is designed for predicting HLA-I binding rather than immunogenicity and thus cannot warrant direct comparison. However, it is a widely used tool in many neoantigen prediction workflows, and as such NetMHCpan was included here only for reference.

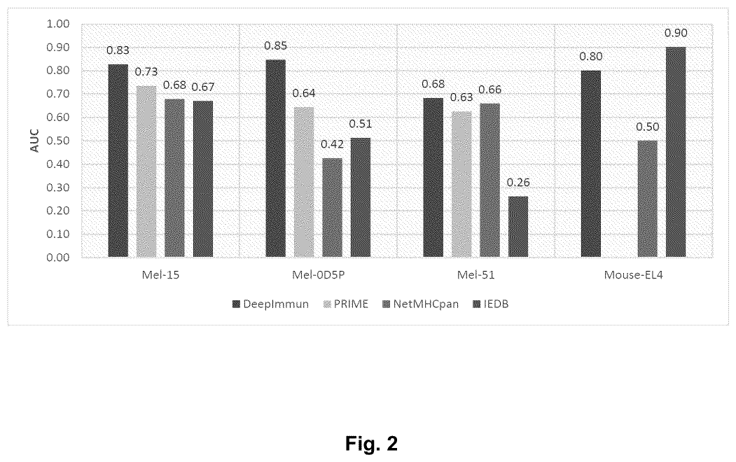

The four prediction tools were evaluated based on two criteria. The first criterion is the area under the receiver operating characteristic curve (ROC-AUC) of their predictions on candidate neoantigens that had been tested in T cell assays of each individual patient. FIG. 2 shows the AUC of the four prediction tools. DeepImmun outperformed the other tools on two patients Mel-15 and Mel-0D5P. On patient Mel-51, DeepImmun, PRIME, and NetMHCpan showed comparable performance. For Mouse-EL4, IEDB predictor achieved the highest AUC of 90%, followed by DeepImmun and NetMHCpan (PRIME does not support mouse data). The unusually higher performance of the IEDB predictor on mouse data than on human data may be because Mouse-EL4 contains two well-studied alleles, H2-Db and H2-Kb, which were included in IEDB training data. Overall, DeepImmun achieved an average AUC of 79% per dataset. The relative performance between the four predictions tools also reflected the characteristics of their underlying models: DeepImmun is a personalized model; PRIME is an allele-specific model; IEDB predictor is a general model trained on a limited dataset; and NetMHCpan is a HLA-binding prediction model.

The main purpose of immunogenicity prediction is to prioritize and select candidate neoantigens to reduce the time and costs of in vitro validations. Thus, the second evaluation criterion is the ability to rank neoantigens among potentially mutated peptides identified from an individual patient. Mutated peptides, including neoantigens, cannot be found in the standard Swiss-Prot human protein database. Thus, a de novo sequencing workflow was first applied to the MS data of the patient to identify new peptides that were not in the database. The four prediction tools were then applied to rank those candidate peptides, including mutated peptides and the neoantigens. They were expected to rank the neoantigens at the top of the list of candidate peptides. It's worth noting that the present MS-based approach only considered candidate peptides identified from MS data, while other genomic approaches often considered candidate peptides translated from genomic sequences.

Table 2 shows how the four prediction tools ranked five neoantigens among 3638 candidate peptides identified by de novo sequencing on the MS data of patient Mel-15's tumor tissues. Notably, the first two neoantigens, KLILWRGLK and RLFLGLAIK, were ranked within the top 1% by DeepImmun, while the next two, GRIAFFLKY and RTYSLSSALR, were ranked within the top 1.5% and 15%, respectively. The last neoantigen, SQIILRQH, should be interpreted with caution: it was identified and tested by Wilhelm et al. as positive; however, its NetMHCpan % Rank is 11.1%, indicating that it may be not likely to be presented by HLA molecules. Overall, DeepImmun outperformed the other tools in this ranking evaluation. Full raw list of peptides, their scores and ranks not included.

Table 2 (below) shows Performance evaluation of four immunogenicity prediction tools on three individual patients and one mouse cancer cell line with Predicted ranks of immunogenic neoantigens among candidate peptides identified by de novo sequencing of MS data of patient Mel-15. (IEDB refers to the immunogenicity prediction tool on IEDB website developed by Calis et al., 2013. Bold underlined letters indicate mutated amino acids. MS: Mass Spectrometry).

| | Predicted ranks | | | |
|---|---|---|---|---|
| Neoantigens | DeepImmun | PRIME | NetMHCpan | IEDB |
| Mel-15 | | | | |
| (5 neoantigens/ | | | | |
| 3638 candidates) | | | | |
| KLILWRGLK | 33 | 1325 | 2140 | 43 |
| RLFLGLAIK | 36 | 818 | 906 | 401 |
| GRIAFFLKY | 53 | 125 | 355 | 349 |
| RTYSLSSALR | 545 | 1896 | 1782 | 3520 |
| SQIILRQH | 1902 | 3635 | 3634 | 661 |

In summary, evaluation results on three cancer patients and one mouse cancer cell line and showed that the personalized models described herein achieved an average accuracy of 79% and outperformed existing immunogenicity prediction tools. Furthermore, the models were able to rank neoantigens that elicited CD8+ T-cell responses within the top 15% of candidate peptides identified from the patients, thus reducing the time and costs of complicated in vitro validations.

Discussion In this study, DeepImmun, a personalized model for immunogenicity prediction was proposed by resembling the negative and positive selections of CD8+ T cells in an individual patient. The model used HLA-I self peptides derived from MS-based immunopeptidomics of the patient as negative training data and allele-matched positive T cell epitopes from the IEDB as positive training data. It was shown that DeepImmun achieved an average accuracy of 79% on four individual patients and outperformed existing tools. More importantly, DeepImmun was able to rank immunogenic neoantigens within the top 15% of candidate de novo peptides identified from the MS data of the patient tumor tissues, thus reducing the time and costs for further in vitro validations.

The present approach requires HLA-I self peptides derived from MS-based immunopeptidomics of an individual patient for training and experimentally validated neoantigens of the same patient for evaluation. Although this kind of individual patient's data is limited and only four such datasets were found for the analysis in this study, results obtained nonetheless demonstrated the validity and the benefits of the personalized approach described herein to model the negative and positive selections of CD8+ T cells for immunogenicity prediction. The present approach is based on MS immunopeptidomics, which has yet to see wide adoption in the industry. As such, the present inventors have developed models, systems, and processes, providing an alternative and improved MS immunopeptidomics approach to existing approaches.

### Example 2—STAR Method Details

Preparation of training data for each patient. HLA-I peptides were retrieved from HLA-I immunoprecipitation assays followed by MS experiments on the patient's sample. In this paper, MS data was re-analyzed from previous studies and did not perform the HLA or MS experiments ourselves. The MS data was searched against the standard Swiss-Prot human protein database (version Jun. 15, 2020) using PEAKS Xpro (version 10.6) with no-enzyme-specific cleavage and FDR of 1%. Precursor mass and fragment ion mass tolerances were set as 15.0 ppm and 0.05 Da, respectively; M(Oxidation) and NQ(Deamidation) were set as variable modifications. The identified peptides were subsequently checked for their HLA-I characteristic length distribution; those with lengths <8 or >14 amino acids were filtered out. The resulting peptides formed the negative training data. Note that, ideally, those HLA-I self peptides should be identified from a normal sample, but for a tumor sample that contains both normal and tumor cells, this approach still works because the database search uses standard human proteins without any tumor mutations. There might be an elevated number of peptides coming from tumor-associated proteins, but they are still considered as self peptides.

Table of T cell assays was downloaded from IEDB with the following filters: linear epitopes, HLA class I, and host as human or mouse. For each individual patient, epitopes were selected if they matched the patient's HLA-I alleles and had positive Assay Qualitative Measure (including Positive, Positive-High, Positive-Intermedia, Positive-Low). Epitopes with lengths <8 or >14 amino acids were filtered out. The resulting peptides formed the positive training data. Full raw list of training data not included.

Bidirectional LSTM network and model training. The model implementation was done in the TensorFlow framework (version 2.6). Amino acid letters were converted to integer indices and peptide sequences were padded with 0 till a maximum length of 15. Keras Sequential model was used that consists of an embedding layer of 8 neural units, a bi-directional LSTM layer of 8 units, a fully-connected layer with L2 regularizer, and a sigmoid activation layer. The model was trained using Adam optimizer and binary cross entropy loss for 100 epochs, only model weights with the best validation loss were saved.

For each individual patient, the training data was splitted into three sets train-validation-test with ratio 80%-10%-10% and no overlapping peptides. As the number of negative peptides was often several times higher than the number of positive peptides, downsampling was performed on negative peptides and trained 100 ensemble models per individual patient. The ensemble models were sorted according to their performance on the validation set and the average of the top-10 models was selected as the final prediction model for that patient.

The trained model is named as DeepImmun. For an input peptide sequence, DeepImmun outputs a score from 0 to 1, a higher score indicates that the input peptide is more likely to be recognized by CD8+ T cells of a particular patient. Since the model is personalized, its prediction should be interpreted only in the context of the patient whose personal data was used to train the model.

Evaluation of immunogenicity prediction tools. DeepImmun, PRIME (version 1.0), NetMHCpan (version 4.1), and IEDB immunogenicity predictor were evaluated on the neoantigens that had been validated by T cell assays on three cancer patients and one mouse cancer cell line. The evaluation data is provided in Supplementary Table S2. For each individual patient, the evaluation data had been carefully excluded from the training data before the training process.

The four prediction tools were evaluated based on two criteria. The first criterion is the area under the receiver operating characteristic curve (ROC-AUC) of their predictions on candidate neoantigens tested in T cell assays. DeepImmun score is a value from 0 to 1 (explained above). For PRIME and NetMHCpan, their % Rank scores was used, which represent the rank of the predicted score compared to a set of random natural peptides (as suggested by the tools' authors). IEDB immunogenicity predictor's score is mainly a log enrichment score, often from −1 to 1; a higher score indicates a higher chance that the peptide is immunogenic. The tools' predicted scores and the T cell responses of tested neoantigens were used to calculate the ROC-AUC with scikit-learn.

The second criterion is the tools' ranking of neoantigens among potentially mutated peptides identified from each individual patient. Mutated peptides, including neoantigens, cannot be found in the standard Swiss-Prot human protein database. Thus, a de novo sequencing workflow was first applied to the MS data to identify new peptides that were not in the database. Precursor mass and fragment ion mass tolerances were set as 15.0 ppm and 0.05 Da, respectively; M(Oxidation) and NQ(Deamidation) were set as variable modifications. A second-round database search using PEAKS Xpro was performed to select de novo peptides with FDR 1%. The identified peptides were further filtered by length and HLA-I binding, only those with 8-14 amino acids and NetMHCpan % Rank <2% were selected. Those filters were to ensure high-confidence identifications and proper HLA characteristics. The four prediction tools were then applied to rank those candidate peptides, including the neoantigens; they were expected to rank the neoantigens at the top of the list of candidate peptides for each individual patient.

Although the embodiments have been described in detail, it should be understood that various changes, substitutions and alterations can be made herein. Moreover, the scope of the present application is not intended to be limited to the particular embodiments or examples described in the specification. As can be understood, the examples described above and illustrated are intended to be exemplary only.

For example, the present invention contemplates that any of the features shown in any of the embodiments described herein, may be incorporated with any of the features shown in any of the other embodiments described herein, and still fall within the scope of the present invention.

### REFERENCES

1. Hu, Z., Ott, P. A. & Wu, C. J. Towards personalized, tumour-specific, therapeutic vaccines for cancer. Nat. Rev. Immunol. 18, 168-182 (2018).
2. The problem with neoantigen prediction. Nat. Biotechnol. 35, 97 (2017).
3. Vitiello, A. & Zanetti, M. Neoantigen prediction and the need for validation. Nat. Biotechnol. 815-817 (2017).
4. Tran, N. H., Xu, J. & Li, M. A tale of solving two computational challenges in protein science: neoantigen prediction and protein structure prediction. Brief. Bioinform. 23, (2022).
5. Bassani-Sternberg, M. et al. Direct identification of clinically relevant neoepitopes presented on native human melanoma tissue by mass spectrometry. Nat. Commun. 7, 13404 (2016).
6. Bulik-Sullivan, B. et al. Deep learning using tumor HLA peptide mass spectrometry datasets improves neoantigen identification. Nat. Biotechnol. (2018) doi:10.1038/nbt.4313.
7. Reynisson, B., Alvarez, B., Paul, S., Peters, B. & Nielsen, M. NetMHCpan-4.1 and NetMHCllpan-4.0: improved predictions of MHC antigen presentation by concurrent motif deconvolution and integration of MS MHC eluted ligand data. Nucleic Acids Res. 48, W449-W454 (2020).
8. Bassani-Sternberg, M. et al. Deciphering HLA-I motifs across HLA peptidomes improves neo-antigen predictions and identifies allostery regulating HLA specificity. PLoS Comput. Biol. 13, e1005725 (2017).
9. Sarkizova, S. et al. A large peptidome dataset improves HLA class I epitope prediction across most of the human population. Nat. Biotechnol. 38, 199-209 (2020).
10. Racle, J. et al. Robust prediction of HLA class II epitopes by deep motif deconvolution of immunopeptidomes. Nat. Biotechnol. 37, 1283-1286 (2019).
11. Xin, L. et al. A streamlined platform for analyzing tera-scale DDA and DIA mass spectrometry data enables highly sensitive immunopeptidomics. Nat. Commun. 13, 1-9 (2022).
12. Calis, J. J. A. et al. Properties of MHC class I presented peptides that enhance immunogenicity. PLoS Comput. Biol. 9, e1003266 (2013).
13. Chowell, D. et al. TCR contact residue hydrophobicity is a hallmark of immunogenic CD8+ T cell epitopes. Proc. Natl. Acad. Sci. U.S.A 112, E1754-62 (2015).
14. Schmidt, J. et al. Prediction of neo-epitope immunogenicity reveals TCR recognition determinants and provides insight into immunoediting. Cell Rep Med 2, 100194 (2021).
15. Wells, D. K. et al. Key Parameters of Tumor Epitope Immunogenicity Revealed Through a Consortium Approach Improve Neoantigen Prediction. Cell 183, 818-834.e13 (2020).
16. Richman, L. P., Vonderheide, R. H. & Rech, A. J. Neoantigen Dissimilarity to the Self-Proteome Predicts Immunogenicity and Response to Immune Checkpoint Blockade. Cell Syst 9, 375-382.e4 (2019).
17. Springer, I., Besser, H., Tickotsky-Moskovitz, N., Dvorkin, S. & Louzoun, Y. Prediction of Specific TCR-Peptide Binding From Large Dictionaries of TCR-Peptide Pairs. Front. Immunol. 11, 1803 (2020).
18. Montemurro, A. et al. NetTCR-2.0 enables accurate prediction of TCR-peptide binding by using paired TCRα and β sequence data. Commun Biol 4, 1060 (2021).
19. Fischer, D. S., Wu, Y., Schubert, B. & Theis, F. J. Predicting antigen specificity of single T cells based on TCR CDR3 regions. Mol. Syst. Biol. 16, e9416 (2020).
20. Dhusia, K., Su, Z. & Wu, Y. A structural-based machine learning method to classify binding affinities between TCR and peptide-MHC complexes. Mol. Immunol. 139, 76-86 (2021).
21. Morel, P. A., Faeder, J. R., Hawse, W. F. & Miskov-Zivanov, N. Modeling the T cell immune response: a fascinating challenge. J. Pharmacokinet. Pharmacodyn. 41, 401-413 (2014).
22. Hogquist, K. A., Baldwin, T. A. & Jameson, S. C. Central tolerance: learning self-control in the thymus. Nat. Rev. Immunol. 5, 772-782 (2005).
23. Tran, N. H. et al. Deep learning enables de novo peptide sequencing from data-independent-acquisition mass spectrometry. Nat. Methods 16, 63-66 (2019).
24. Tran, N. H., Zhang, X., Xin, L., Shan, B. & Li, M. De novo peptide sequencing by deep learning. Proc. Natl. Acad. Sci. U.S.A 114, 8247-8252 (2017).
25. Wilhelm, M. et al. Deep learning boosts sensitivity of mass spectrometry-based immunopeptidomics. Nat. Commun. 12, 3346 (2021).
26. Zhou, X.-X. et al. pDeep: Predicting MS/MS Spectra of Peptides with Deep Learning. Anal. Chem. 89, 12690-12697 (2017).
27. Ma, C. et al. Improved Peptide Retention Time Prediction in Liquid Chromatography through Deep Learning. Anal. Chem. 90, 10881-10888 (2018).
28. Tran, N. H., Zhang, X. & Li, M. Deep Omics. Proteomics 18, (2018).
29. Chong, C. et al. Integrated proteogenomic deep sequencing and analytics accurately identify non-canonical peptides in tumor immunopeptidomes. Nat. Commun. 11, 1293 (2020).
30. Kalaora, S. et al. Identification of bacteria-derived HLA-bound peptides in melanoma. Nature 592, 138-143 (2021).
31. Laumont, C. M. et al. Noncoding regions are the main source of targetable tumor-specific antigens. Sci. Transl. Med. 10, (2018).
32. Tran, N. H. et al. Personalized deep learning of individual immunopeptidomes to identify neoantigens for cancer vaccines. Nature Machine Intelligence 2, 764-771 (2020).
33. Developers, T. TensorFlow. (2022). doi:10.5281/zenodo.6574269.
34. Pedregosa, F. et al. Scikit-learn: Machine Learning in Python. J. Mach. Learn. Res. 12, 2825-2830 (2011).

What is claimed is:

1. A computer-implemented method for personalized identification of neoantigens from sample de novo peptides sequences obtained from a patient, the method comprising:
obtaining a first dataset of HLA-1 binding de novo self peptides sequences of the patient;
obtaining a second dataset of patient allele-matched T-cell epitope sequences; wherein the first and second datasets are for training an artificial neural network to classify the sample peptide sequences based on T-cell recognition;
selecting sample peptide sequences that match with sequences of the second dataset; and
excluding sample peptide sequences that match with the first dataset, wherein the remaining selected sample peptide sequences are identified as candidate neoantigens.

2. The method of claim 1, wherein obtaining the first dataset comprises:
conducting a HLA-1 immunoprecipitation assay on a patient cell sample; and
sequencing peptides from the immunoprecipitation assay using mass spectrometry.

3. The method of claim 2, comprising obtaining sequenced peptides that are between and including 8 and 14 amino acids in length for the first dataset.

4. The method of claim 2, wherein the patient cell sample comprise a normal cell sample, or a combination of normal and tumor cells sample.

5. The method of claim 1, wherein obtaining the second dataset comprises:
obtaining a database of epitopes that are T cell positive, and
selecting epitopes from the database that match against the patient's HLA-1 alleles.

6. The method of claim 5, comprising selecting peptides that are between and including 8 and 14 amino acids in length for the second dataset.

7. The method of claim 1, comprising training a binary classification model to predict T cell response to the sample peptide sequences.

8. The method of claim 1, comprising outputting a score representing the likelihood that a candidate neoantigen will be recognized by CD8+ T cells of the patient.

9. A computer implemented system for personalized identification of neoantigens from sample peptides sequences obtained from a patient using neural networks, the computer implemented system comprising:
a processor and at least one memory providing a plurality of layered nodes configured to form an artificial neural network for generating a probability measure for one or more neoantigen candidates, the artificial neural network trained on:
a first dataset of HLA-1 binding self peptides of the patient, and
a second dataset of patient allele-matched T-cell epitopes,
to classify the sample peptide sequences based on T-cell recognition, wherein the plurality of layered nodes receives a peptide sequence as input;
the processor configured to:
select sample peptide sequences that match with sequences of the second dataset, and
exclude sample peptide sequences that match with the first dataset, wherein the remaining selected sample peptide sequences are identified as candidate neoantigens.

10. The system of claim 9, wherein the processor is configured to output a score representing the likelihood that a candidate neoantigen will be recognized by CD8+ T cells of the patient.

11. The system of claim 9, wherein the first and second dataset comprise peptide sequences that are between and including 8 and 14 amino acids in length.

12. The system of claim 9, wherein the artificial neural network is trained on a binary classification model to predict T cell response to the sample peptide sequences.

13. The system of claim 9, wherein the plurality of layered nodes comprise one or more of:
an embedding layer;
a bi-directional LSTM layer;
a fully-connected layer with L2 regularizer; and
a sigmoid activation layer.

14. The system of claim 13, wherein the plurality of layered nodes comprise one or more of:
an embedding layer of 8 neural units;
a bi-directional LSTM layer of 8 units;
a fully-connected layer with L2 regularizer; and
a sigmoid activation layer.
